(19) United States
(12) Patent Application Publication
(10) Pub. No.: US 2022/0291222 A1
(43) Pub. Date: Sep. 15, 2022

(54) EXTRACELLULAR VESICLE BIOMARKERS FOR PANCREATIC CANCER DETECTION, DIAGNOSIS AND MONITORING

(71) Applicant: Tymora Analytical Operations, Inc., West Lafayette, IN (US)

(72) Inventors: Anton Ilyuk, West Lafayette, IN (US); Weiguo A. Tao, West Lafayette, IN (US); BRUNO BOCKORNY, West Lafayette, IN (US); LING HUANG, West Lafayette, IN (US); LAKSHMI MUTHUSWAMY, West Lafayette, IN (US); MANUEL HIDALGO, West Lafayette, IN (US); SENTHIL MUTHUSWAMY, West Lafayette, IN (US)

(21) Appl. No.: 17/692,377

(22) Filed: Mar. 11, 2022

Related U.S. Application Data
(60) Provisional application No. 63/159,573, filed on Mar. 11, 2021.

(51) Int. Cl. G01N 33/574

(52) U.S. Cl. CPC G01N 33/57438; G01N 33/57488

(57) ABSTRACT

Methods and products for the identification and detection of new pancreatic cancer biomarkers based on proteins in biofluids, such as plasma and serum extracellular vesicles.

### CROSS-REFERENCE TO RELATED APPLICATION

This U.S. patent application claims priority to U.S. Provisional Application No. 63/159,573 filed Mar. 11, 2021, to the above-named inventors, the disclosure of which is considered part of the disclosure of this application and is hereby incorporated by reference in its entirety.

### FEDERALLY SPONSORED RESEARCH OR DEVELOPMENT

This study was made with government support under Grant No. CA213863 awarded by the National Institutes of Health. The government has certain rights in the outcome of this study.

### SEQUENCE LISTING, A TABLE, OR A COMPUTER PROGRAM

Not applicable.

### FIELD OF THE INVENTION

This invention relates generally to a method to isolate proteins from biofluids, such as plasma and serum, for biomarker discovery or for clinical detection. More particularly, this invention relates to non-invasive early disease diagnosis, disease monitoring and disease classification. In one aspect, this invention relates to unique proteins capable of differentiating pancreatic cancer plasma or serum samples from healthy samples and non-cancer conditions control samples. In another aspect, this invention relates to early-stage detection of pancreatic cancer by plasma or serum test.

### BACKGROUND

The rates of survival for pancreatic cancer are very low, primarily because the cancer is detected at a very late stage. It is critical to develop a non-invasive screening test for early detection of pancreatic cancer, which would significantly improve disease prognosis and survival rates.

Pancreatic ductal adenocarcinoma (PDAC) has the worst prognosis of all common tumors. The vast majority of patients are diagnosed when curative surgery is no longer possible (Rahib et al. 2014; Spanknebel and Conlon 2001). Earlier cancer diagnosis could increase survival rates by an estimated 5-fold and more reliable and real-time assessment of treatment effects in patients with cancer could improve quality of life and reduce healthcare costs (Ghatnekar et al. 2013).

Chronic pancreatitis and intraductal papillary mucinous neoplasm (IPMN) are established risk factors for PDAC accounting for up to 20% of the cases. Surveillance strategies for this heterogenous population is not standardized and patients with “worrisome” imaging features, particularly those with high risk IPMN, may undergo surgical resection. Unfortunately, the imaging criteria reflexing patients to surgery are imperfect, leading to both over- and under-treatment.

The gold standard for diagnosis of pancreatic cancer, particularly in those with underlying pancreatic diseases (cysts or pancreatitis) is the endoscopic ultrasound (EUS) with fine needle biopsy, which has a sensitivity of 90% and specificity of 72% (Li et al. 2014). The main limitations of the EUS are: (1) it is a relatively invasive procedure; (2) limited availability, needs to be performed in a center with high volume as the diagnostic accuracy is directly related to the operator's skills; (3) elevated costs, ranging from $2,152 to $2,605 per procure (Aadam et al. 2016).

The current blood-based biomarker that is clinically available (CA19-9) suffers from poor sensitivity (75.4%) and specificity for the diagnosis of pancreatic cancer (77.6%) (Ghatnekar et al. 2013). CA19-9 can be elevated in non-malignant etiologies such as jaundice and biliary obstruction. For patients with established diagnosis of pancreatic cancer, CA19-9 elevates late in the disease course making it unsuitable for detection of early-stage disease, when curative treatment options are available.

There are no credentialed biomarkers with high enough performance to assist in therapeutic stratification. Various approaches to achieving this are being explored, including circulating tumor cells (CTCs), metabolites, proteomics, cell-free DNA and circulating extracellular vesicles (EVs) (Duffy et al. 2010; Kelly et al. 2015; Nagrath et al. 2016).

Extracellular vesicles (exosomes and microvesicles) are nanosized particles released by most cell types and involved in intercellular communication, including transmission of oncogenic and inflammatory signals (Costa-Silva et al. 2015). Their exoDNA, exoRNA and protein profiles highly reflect parental cells, therefore offering an attractive strategy for diagnosing cancers using “liquid biopsies” (Melo et al. 2015; Costa-Silva et al. 2015). Cancer cells can shed a much higher concentration of EVs per cell, making them substantially more abundant than CTCs (Taylor and Gercel-Taylor 2008; Riches et al. 2014). EVs are also structurally more stable than soluble proteins, cell-free DNA and metabolites, underscoring their potential as serologic biomarkers (Thery 2015; Costa-Silva et al. 2015).

However, the application of EV biomarkers in clinic has been hampered by two practical challenges:

(1) Prior discovery EV proteomics have been done in vitro using cell lines, which are not representative of the heterogeneity of human PDAC and are unable to recapitulate the systemic/inflammatory response to cancer. Therefore, those prior EV signatures lacked performance to distinguish PDAC from high-risk chronic pancreatic diseases (Yang et al. 2017; Castillo et al. 2018; Madhavan et al. 2015) which is critical in clinical practice.

(2) Traditional workflow for EV isolation directly from blood samples require laborious techniques (such as ultracentrifugation) that are not scalable for clinical use.

There is a critical need for a new diagnostic test that would avoid these drawbacks and enable more effective non-invasive detection of pancreatic cancer at the earliest stage, which would significantly improve disease prognosis and survival rates, particularly in those with underlying diseases of the pancreas where detection of cancer is very challenging.

In one aspect, this disclosure is related to a robust method for the identification and detection of new biomarkers based on proteins for pancreatic cancer, for the purposes of disease diagnosis, prognosis, detection, monitoring, patient stratification, drug response analysis, therapy selection, or the like.

The proposed method introduces a novel platform technology to isolate proteins from biofluids, such as plasma and serum, for biomarker discovery or for clinical detection.

In another aspect, this disclosure is related to a method that successfully demonstrates the feasibility of developing biofluid-derived EV proteins for cancer profiling. It has tremendous transformative potential for early cancer diagnosis, monitoring and classification based on actual activated pathways using plasma and serum as the source.

Furthermore, once fully established, these new biomarkers can be employed either isolated or as part of a panel of biomarkers as a liquid biopsy in clinical scenarios: (1) as a surveillance test in high-risk patients, such as those with high-risk cystic diseases, hereditary risk of cancer, among others or (2) as a liquid biopsy for the longitudinal monitoring of treatment response in patients with already established cancer diagnosis.

In yet another aspect, this disclosure relates to a biomarker panel for detection and monitoring of pancreatic cancer.

Still further, it is envisioned to further apply this innovative procedure to validate and fully develop pre-determined biomarkers panels.

### BRIEF SUMMARY OF THE INVENTION

The invention now will be described more fully hereinafter with reference to the accompanying drawings, which are intended to be read in conjunction with both this summary, the detailed description and any preferred and/or particular embodiments specifically discussed or otherwise disclosed. This invention may, however, be embodied in many different forms and should not be construed as limited to the embodiments set forth herein; rather, these embodiments are provided by way of illustration only and so that this disclosure will be thorough, complete and will fully convey the full scope of the invention to those skilled in the art.

This disclosure is related to a method for the identification and detection of new biomarkers based on proteins—a true measure of dynamic activity and cellular signaling. The proposed method introduces a novel platform technology to isolate extracellular vesicles (EV) proteins from biofluids such as plasma and serum for biomarker discovery and clinical detection of pancreatic cancer.

The method for capture, enrichment or isolation of extracellular vesicles is selected from the group consisting of Extracellular Vesicles total recovery and purification (EVtrap™), ultracentrifugation (UC), filtrations, antibody-based purification, size-exclusion approach, polymer precipitation and affinity capture.

This disclosure is the first such method to successfully demonstrate the feasibility of developing plasma- and serum-derived EV proteins for pancreatic cancer detection and profiling. It is envisioned to further apply this innovative procedure to validate and fully develop the disclosed current biomarker panel in Table I for detection and monitoring of pancreatic cancer.

### BRIEF DESCRIPTION OF THE DRAWINGS

The above-mentioned and other features of this disclosure, and the manner of attaining them, will become more apparent and the disclosure itself will be better understood by reference to the following description of embodiments of the disclosure taken in conjunction with the accompanying drawings, wherein:

FIG. 1 is the graphical illustration of principal component (PC) analysis on protein abundance in EVs present in the plasma of patients with PDAC (N=93), chronic pancreatitis (N=12), IPMN (N=8) and healthy controls (N=11) showing distinct EV proteome clustering.

FIG. 2 illustrates a summary of select EV Biomarkers Discovered.

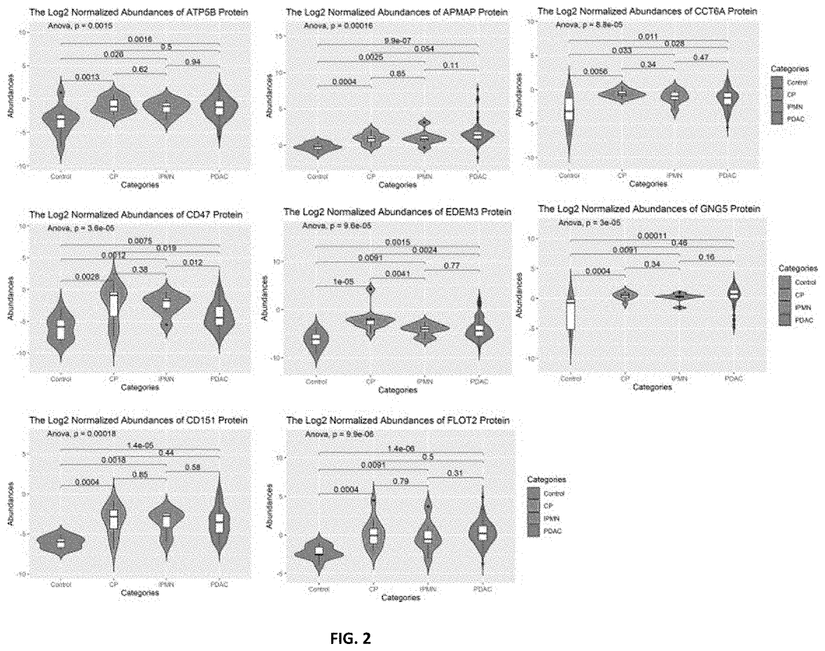

FIG. 3 illustrates a summary of select EV Biomarkers Discovered.

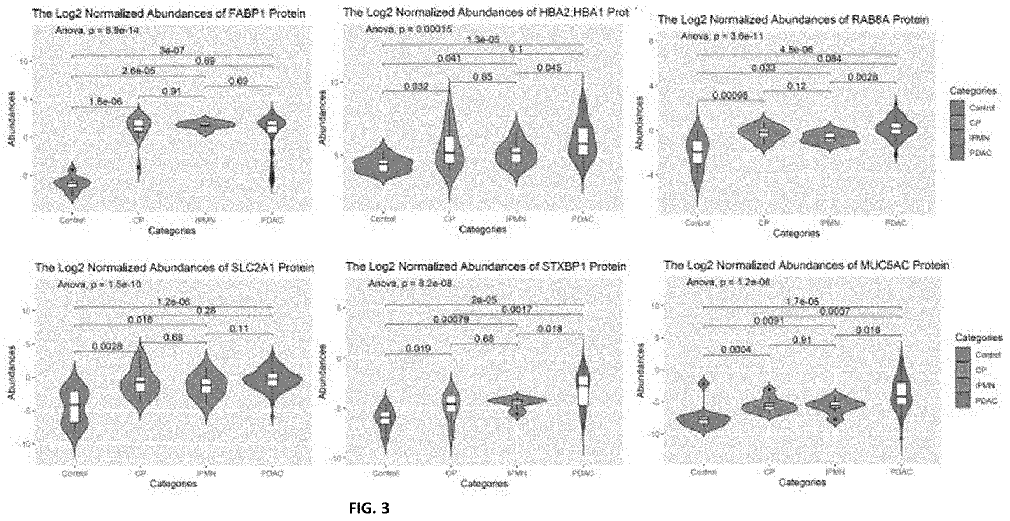

FIG. 4 illustrates a summary of select EV Biomarkers Discovered.

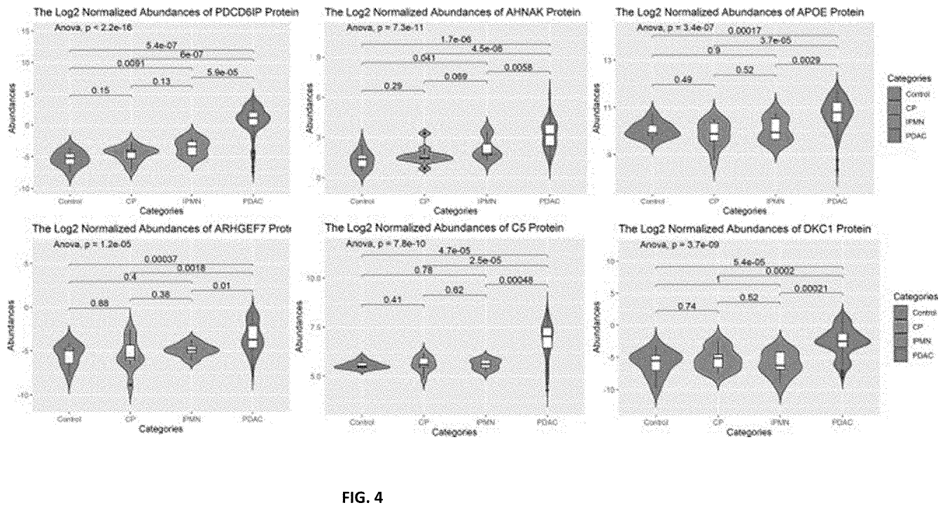

FIG. 5 illustrates a summary of select EV Biomarkers Discovered.

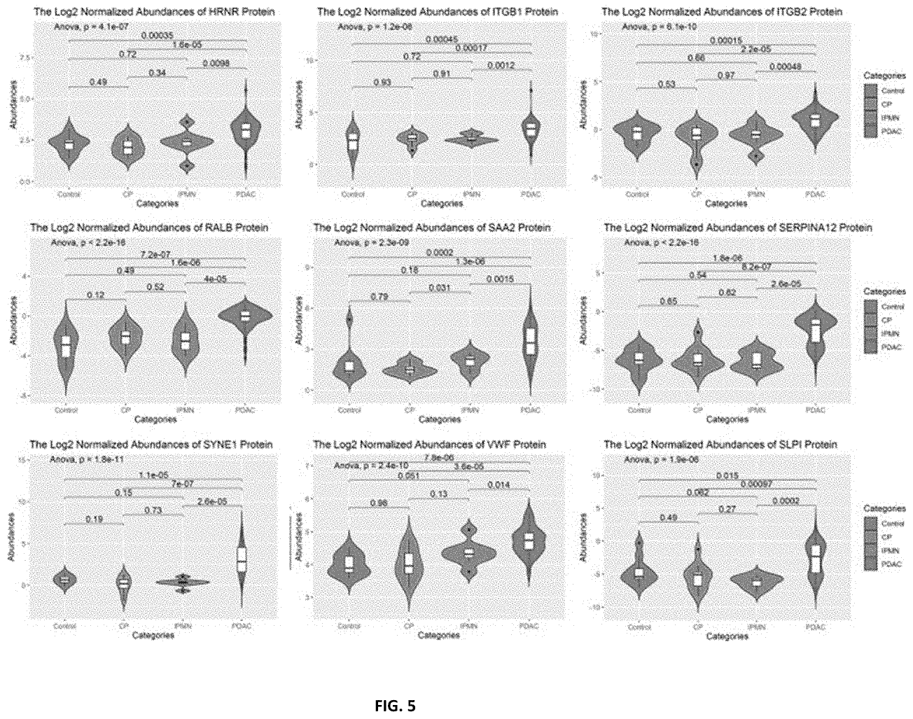

FIG. 6 illustrates a summary of select EV Biomarkers Discovered.

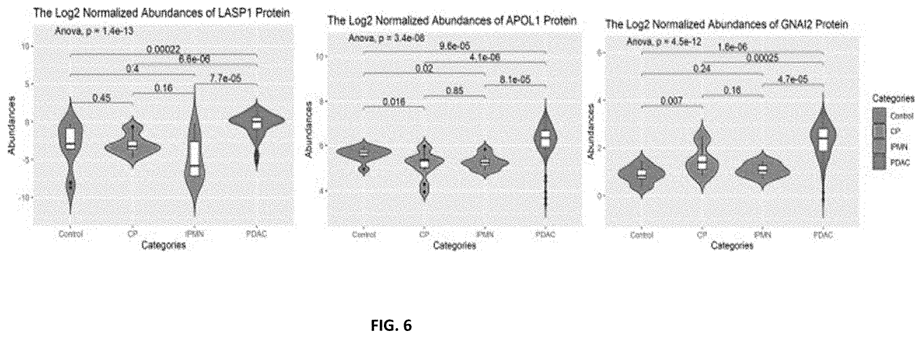

### DETAILED DESCRIPTION OF THE INVENTION

The following detailed description includes references to the accompanying drawings, which forms a part of the detailed description. The drawings show, by way of illustration, specific embodiments in which the invention may be practiced. These embodiments, which are also referred to herein as “examples,” are described in enough detail to enable those skilled in the art to practice the invention. The embodiments may be combined, other embodiments may be utilized, or structural, and logical changes may be made without departing from the scope of the present invention. The following detailed description is, therefore, not to be taken in a limiting sense.

Before the present invention of this disclosure is described in such detail, however, it is to be understood that this invention is not limited to particular variations set forth and may, of course, vary. Various changes may be made to the invention described and equivalents may be substituted without departing from the true spirit and scope of the invention. In addition, many modifications may be made to adapt a particular situation, material, composition of matter, process, process act(s) or step(s), to the objective(s), spirit or scope of the present invention. All such modifications are intended to be within the scope of the disclosure made herein.

Unless otherwise indicated, the words and phrases presented in this document have their ordinary meanings to one of skill in the art. Such ordinary meanings can be obtained by reference to their use in the art and by reference to general and scientific dictionaries.

References in the specification to “one embodiment” indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The following explanations of certain terms are meant to be illustrative rather than exhaustive. These terms have their ordinary meanings given by usage in the art and in addition include the following explanations.

Unless otherwise stated, a reference to a compound or component includes the compound or component by itself, as well as in combination with other compounds or components, such as mixtures of compounds.

As used herein, the term “and/or” refers to any one of the items, any combination of the items, or all of the items with which this term is associated.

As used herein, the singular forms “a,” “an,” and “the” include plural reference unless the context clearly dictates otherwise.

As used herein, the terms “include,” “for example,” “such as,” and the like are used illustratively and are not intended to limit the present invention.

As used herein, the terms “preferred” and “preferably” refer to embodiments of the invention that may afford certain benefits, under certain circumstances. However, other embodiments may also be preferred, under the same or other circumstances.

Furthermore, the recitation of one or more preferred embodiments does not imply that other embodiments are not useful and is not intended to exclude other embodiments from the scope of the invention.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element without departing from the teachings of the disclosure.

All publications, patents and patent applications cited herein, whether supra or infra, are hereby incorporated by reference in their entirety to the same extent as if each individual publication, patent or patent application was specifically and individually indicated to be incorporated by reference.

While the invention has been described above in terms of specific embodiments, it is to be understood that the invention is not limited to these disclosed embodiments. Upon reading the teachings of this disclosure many modifications and other embodiments of the invention will come to mind of those skilled in the art to which this invention pertains, and which are intended to be and are covered by both this disclosure and the appended claims. It is indeed intended that the scope of the invention should be determined by proper interpretation and construction of the appended claims and their legal equivalents, as understood by those of skill in the art relying upon the disclosure in this specification and the attached drawings.

EV biomarkers discovery directly from blood samples:

The preferred method, EVtrap™, was applied for EV enrichment directly from blood samples to isolate EVs from plasma for subsequent liquid chromatography-mass spectrometry analysis. Processing and enrichment of EVs through this method enabled the removal of soluble proteins, retaining vesicle associated proteins which are more stable in circulation and have enhanced signals from cancer tissues. The protein profiles in EV concentrates are different from protein profiles naturally occurring in patient blood.

### Example 1: EV Proteomics Signature Able to Distinguish PDAC from Benign Pancreatic Diseases

The research protocol was designed, and IRB approved (PI: Bockorny) allowing for recruitment of patients with established diagnosed of pancreatic cancer at all stages: patients with underlying non-malignant conditions of the pancreas such as chronic pancreatitis and cystic diseases of the pancreas; as well as healthy patients (controls). All patients provided written informed consent for this study. Clinical data were extracted through medical records and patient interview. Blood samples of 124 patients were collected by the research personnel and subsequently processed and stored in our laboratory. The blood samples were processed using the preferred method, EVtrap™, and subsequent liquid chromatography—mass spectrometry analysis.

In total, EV proteomics was conducted on a cohort of 124 patients including various stages of PDAC (N=93), chronic pancreatitis of different etiologies (N=12), IPMN (N=8) and healthy controls (N=11), wherein, on average, ^{˜}1,000 unique EV proteins were identified from each individual case.

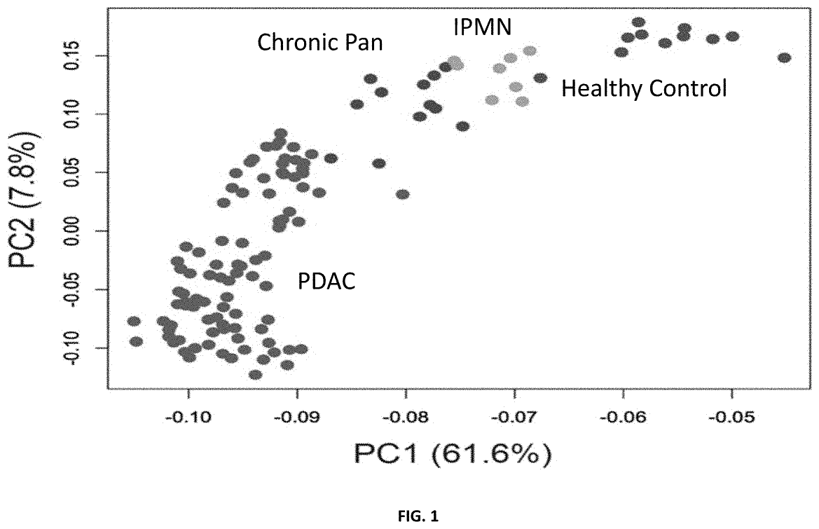

Different biostatistical analysis of the proteomics were performed, and it was observed that the EV secretome of PDAC patients had clear separation from chronic pancreatitis, IPMN and controls (FIG. 1). Notably, EV proteome in PDAC had enrichment for immune-, complement- and coagulation-related proteins, underscoring the power of direct-from-blood EV proteomics to expand the understanding of tumor-host interactions, which cannot be recapitulated by in vitro studies.

The differential expression of individual EV proteins was analyzed. Focusing on EV markers that were enriched in patients with pancreatic cancer as compared to patients with benign disease of the pancreas and normal controls (biomarkers for diagnosis of pancreatic cancer) as well as on the markers more enriched in advanced pancreatic cancer (stage IV) as compared to early stages (stages I/II) as those represent biomarkers that may serve for disease monitoring of tumor burden.

Summary of the EV Biomarkers Discovered is found in Table I. Table I lists a panel of EV proteins with significant overexpression and enrichment in advanced PDAC but not in early stages or controls, including novel biomarkers as well as proteins previously known to have functional or prognostic implications in PDAC (e.g. integrin B2, ezrin) (Yuan et al. 2015; Giulietti et al. 2018; Meng et al. 2010; Kocher et al. 2009).

The markers listed in Table I can be employed either isolated or as part of a panel of biomarkers as a liquid biopsy in different clinical scenarios: (1) as a surveillance test in high-risk patients, such as those with high-risk cystic diseases of the pancreas (e.g. IPMN), hereditary pancreatitis, chronic pancreatitis. (2) or as a liquid biopsy for the longitudinal monitoring of treatment response in patients with already established pancreatic cancer diagnosis.

It is envisioned that secondary validation of the candidate biomarkers listed in Table I might be performed in an independent cohort of patients (different from the patients from the proteomics) by employing an alternative method to identify such proteins of interest (ELISA, western blot, etc.).

TABLE I
Summary of Preprocessed EV Biomarkers Discovered

| Gene | EV Protein Name | Expression Level |
|---|---|---|
| L1TD1 | LINE-1 type transposase domain-containing protein 1 | Up |
| | [OS = Homo sapiens] | |
| PDCD6IP | Programmed cell death 6-interacting protein [OS = Homo | Up |
| | sapiens] | |
| SERPINA12 | Serpin A12 [OS = Homo sapiens] | Up |
| SCIN | ADSEVERIN [OS = Homo sapiens] | Up |
| LASP1 | Isoform 2 of LIM and SH3 domain protein 1 [OS = Homo sapiens] | Up |
| CCT4 | T-complex protein 1 subunit delta [OS = Homo sapiens] | Up |
| CTSA | lysosomal protective protein [OS = Homo sapiens] | Up |
| SLPI | Antileukoproteinase [OS = Homo sapiens] | Up |
| DKC1 | H/ACA ribonucleoprotein complex subunit 4 [OS = Homo | Up |
| | sapiens] | |
| SYNE1 | Isoform 7 of Nesprin-1 [OS = Homo sapiens] | Up |
| NAPA | alpha-soluble nsf attachment protein [OS = Homo sapiens] | Up |
| CRP | C-reactive protein [OS = Homo sapiens] | Up |
| CCT8 | T-complex protein 1 subunit theta [OS = Homo sapiens] | Up |
| KMT2A | Histone-lysine N-methyltransferase 2A [OS = Homo sapiens] | Up |
| ALAD | Delta-aminolevulinic acid dehydratase [OS = Homo sapiens] | Up |
| PDIA6 | Protein disulfide-isomerase A6 [OS = Homo sapiens] | Up |
| CAST | Calpastatin [OS = Homo sapiens] | Up |
| PSMA1 | Proteasome subunit alpha type-1 [OS = Homo sapiens] | Up |
| IGHV6-1 | immunoglobulin heavy variable 6-1 [OS = Homo sapiens] | Up |
| NME1 | Isoform 3 of Nucleoside diphosphate kinase B [OS = Homo | Up |
| | sapiens] | |
| PSME1 | Proteasome activator complex subunit 1 [OS = Homo sapiens] | Up |
| KPNB1 | Importin subunit beta-1 [OS = Homo sapiens] | Up |
| HIST1H2BB | Histone H2B type 1-B [OS = Homo sapiens] | Up |
| SPRR3 | Small proline-rich protein 3 [OS = Homo sapiens] | Up |
| GPX3 | Glutathione peroxidase 3 [OS = Homo sapiens] | Up |
| MPO | Isoform H14 of Myeloperoxidase [OS = Homo sapiens] | Up |
| RALB | Isoform 2 of Ras-related protein Ral-B [OS = Homo sapiens] | Up |
| CDC42 | Cell division control protein 42 homolog [OS = Homo sapiens] | Up |
| IDH2 | Isocitrate dehydrogenase [NADP], mitochondrial [OS = Homo | Up |
| | sapiens] | |
| KIF13B | Kinesin-like protein KIF13B [OS = Homo sapiens] | Up |
| CANX | Calnexin [OS = Homo sapiens] | Up |
| ITIH4 | Isoform 2 of Inter-alpha-trypsin inhibitor heavy chain H4 | Up |
| | [OS = Homo sapiens] | |
| C5orf24 | UPF0461 protein C5orf24 [OS = Homo sapiens] | Up |
| ATP2A2 | Isoform 4 of Sarcoplasmic/endoplasmic reticulum calcium | Up |
| | ATPase 2 [OS = Homo sapiens] | |
| SCGB2A2 | Mammaglobin-A [OS = Homo sapiens] | Up |
| OTC | Ornithine carbamoyltransferase, mitochondrial [OS = Homo | Up |
| | sapiens] | |
| MUC5AC | Mucin-5AC [OS = Homo sapiens] | Up |
| ITPR1 | Isoform 3 of Inositol 1,4,5-trisphosphate receptor type 1 | Up |
| | [OS = Homo sapiens] | |
| GDI2 | Rab GDP dissociation inhibitor beta [OS = Homo sapiens] | Up |
| TGM2 | Protein-glutamine gamma-glutamyltransferase 2 [OS = Homo | Up |
| | sapiens] | |
| S100A10 | Protein S100-A10 [OS = Homo sapiens] | Up |
| SAA2 | Serum amyloid A-2 protein [OS = Homo sapiens] | Up |
| IFITM3 | Interferon-induced transmembrane protein 3 [OS = Homo | Up |
| | sapiens] | |
| ITGB2 | Integrin beta-2 [OS = Homo sapiens] | Up |
| IGKV1-16 | Immunoglobulin kappa variable 1-16 [OS = Homo sapiens] | Up |
| WFDC12 | WAP four-disulfide core domain protein 12 [OS = Homo sapiens] | Up |
| IGHV3-74 | immunoglobulin heavy variable 3-74 [OS = Homo sapiens] | Up |
| ARHGEF7 | Isoform 1 of Rho guanine nucleotide exchange factor 7 | Up |
| | [OS = Homo sapiens] | |
| RAB7A | ras-related protein Rab-7a [OS = Homo sapiens] | Up |
| TPM2 | Isoform 2 of Tropomyosin beta chain [OS = Homo sapiens] | Up |
| IGKV1-8 | immunoglobulin kappa variable 1-8 [OS = Homo sapiens] | Up |
| VMO1 | Vitelline membrane outer layer protein 1 homolog [OS = Homo | Up |
| | sapiens] | |
| BLMH | bleomycin hydrolase [OS = Homo sapiens] | Up |
| YWHAG | 14-3-3 protein gamma [OS = Homo sapiens] | Up |
| STXBP1 | Syntaxin-binding protein 1 [OS = Homo sapiens] | Up |
| GGCT | gamma-glutamylcyclotransferase [OS = Homo sapiens] | Up |
| SPEN | Msx2-interacting protein [OS = Homo sapiens] | Up |
| CYB5R3 | Isoform 2 of NADH-cytochrome b5 reductase 3 [OS = Homo | Up |
| | sapiens] | |
| SAA1 | Serum amyloid A-1 protein [OS = Homo sapiens] | Up |
| RDX | Isoform 5 of Radixin [OS = Homo sapiens] | Up |
| IGKV1-6 | immunoglobulin kappa variable 1-6 [OS = Homo sapiens] | Up |
| RAB35 | Ras-related protein Rab-35 [OS = Homo sapiens] | Up |
| TULP1 | Isoform 2 of Tubby-related protein 1 [OS = Homo sapiens] | Up |
| LGALS3BP | Galectin-3-binding protein [OS = Homo sapiens] | Up |
| CYP2B6 | Cytochrome P450 2B6 [OS = Homo sapiens] | Up |
| INCENP | Inner centromere protein [OS = Homo sapiens] | Up |
| IGHV5-51 | immunoglobulin heavy variable 5-51 [OS = Homo sapiens] | Up |
| AHNAK | Neuroblast differentiation-associated protein AHNAK | Up |
| | [OS = Homo sapiens] | |
| ALDOC | Fructose-bisphosphate aldolase C [OS = Homo sapiens] | Up |
| PDLIM1 | PDZ and LIM domain protein 1 [OS = Homo sapiens] | Up |
| KRT4 | Keratin, type II cytoskeletal 4 [OS = Homo sapiens] | Up |
| HSP90AA1 | Isoform 2 of Heat shock protein HSP 90-alpha [OS = Homo | Up |
| | sapiens] | |
| CTSD | Cathepsin D [OS = Homo sapiens] | Up |
| FCN2 | Ficolin-2 [OS = Homo sapiens] | Up |
| OBSCN | Isoform 5 of Obscurin [OS = Homo sapiens] | Up |
| DENND4A | C-myc promoter-binding protein [OS = Homo sapiens] | Up |
| HLA-A | HLA class 1 histocompatibility antigen, A-11 alpha chain | Up |
| | [OS = Homo sapiens] | |
| OAF | Out at first protein homolog [OS = Homo sapiens] | Up |
| HLA-B | HLA class 1 histocompatibility antigen, B-41 alpha chain | Up |
| | [OS = Homo sapiens] | |
| KIF16B | Kinesin-like protein KIF16B [OS = Homo sapiens] | Up |
| CFHR3 | Complement factor H-related protein 3 [OS = Homo sapiens] | Up |
| C5 | Complement C5 [OS = Homo sapiens] | Up |
| PARVB | Beta-parvin [OS = Homo sapiens] | Up |
| CD53 | Leukocyte surface antigen CD53 [OS = Homo sapiens] | Up |
| EZR | Ezrin [OS = Homo sapiens] | Up |
| DNAH3 | Dynein heavy chain 3, axonemal [OS = Homo sapiens] | Up |
| RAB5C | Ras-related protein Rab-5C [OS = Homo sapiens] | Up |
| PSMB6 | Proteasome subunit beta type-6 [OS = Homo sapiens] | Up |
| IGKV1D-33 | Immunoglobulin kappa variable 1-33 [OS = Homo sapiens] | Up |
| ST13 | Hsc70-interacting protein [OS = Homo sapiens] | Up |
| PCCB | propionyl-CoA carboxylase beta chain, mitochondrial | Up |
| | [OS = Homo sapiens] | |
| FLOT1 | Flotillin-1 [OS = Homo sapiens] | Up |
| KRT18 | Keratin, type I cytoskeletal 18 [OS = Homo sapiens] | Up |
| GSTP1 | Glutathione S-transferase P [OS = Homo sapiens] | Up |
| SLC9A3R1 | Na(+)/H(+) exchange regulatory cofactor NHE-RF1 [OS = Homo | Up |
| | sapiens] | |
| ADIPOQ | adiponectin [OS = Homo sapiens] | Up |
| ITGAM | Integrin alpha-M [OS = Homo sapiens] | Up |
| DSG1 | Desmoglein-1 [OS = Homo sapiens] | Up |
| APOL1 | Isoform 2 of Apolipoprotein L1 [OS = Homo sapiens] | Up |
| BASP1 | Brain acid soluble protein 1 [OS = Homo sapiens] | Up |
| FETUB | Fetuin-B [OS = Homo sapiens] | Up |
| INPP1 | Inositol polyphosphate 1-phosphatase [OS = Homo sapiens] | Up |
| SF3B1 | splicing factor 3B subunit 1 [OS = Homo sapiens] | Up |
| IGHD | immunoglobulin delta heavy chain [OS = Homo sapiens] | Up |
| SLC25A5 | ADP/ATP translocase 2 [OS = Homo sapiens] | Up |
| HAL | Histidine ammonia-lyase [OS = Homo sapiens] | Up |
| CARD14 | Caspase recruitment domain-containing protein 14 [OS = Homo sapiens] | Up |
| PSMA2 | Proteasome subunit alpha type-2 [OS = Homo sapiens] | Up |
| MMRN2 | Multimerin-2 [OS = Homo sapiens] | Up |
| TNFRSF1A | Isoform 5 of Tumor necrosis factor receptor superfamily | Up |
| | member 1A [OS = Homo sapiens] | |
| IGKV3D-15 | Immunoglobulin kappa variable 3D-15 [OS = Homo sapiens] | Up |
| GNAI2 | guanine nucleotide-binding protein G(i) subunit alpha-2 | Up |
| | [OS = Homo sapiens] | |
| POF1B | Protein POF1B [OS = Homo sapiens] | Up |
| LBP | lipopolysaccharide-binding protein [OS = Homo sapiens] | Up |
| FCGBP | IgGFc-binding protein [OS = Homo sapiens] | Up |
| CPS1 | Carbamoyl-phosphate synthase [ammonia], mitochondrial | Up |
| | [OS = Homo sapiens] | |
| ITGB1 | Isoform 5 of Integrin beta-1 [OS = Homo sapiens] | Up |
| ZNF598 | E3 ubiquitin-protein ligase ZNF598 [OS = Homo sapiens] | Up |
| TIGD4 | Tigger transposable element-derived protein 4 [OS = Homo | Up |
| | sapiens] | |
| HRNR | Hornerin [OS = Homo sapiens] | Up |
| SVEP1 | Sushi, von Willebrand factor type A, EGF and pentraxin domain- | Up |
| | containing pr | |
| PRDX5 | Peroxiredoxin-5, mitochondrial [OS = Homo sapiens] | Up |
| PLEC | Isoform 7 of Plectin [OS = Homo sapiens] | Up |
| CASP14 | Caspase-14 [OS = Homo sapiens] | Up |
| HNRNPA2B1 | heterogeneous nuclear ribonucleoproteins A2/B1 [OS = Homo | Up |
| | sapiens] | |
| IGFALS | Insulin-like growth factor-binding protein complex acid labile | Up |
| | subunit | |
| | [OS = Homo sapiens] | |
| ENDOD1 | endonuclease domain-containing 1 protein [OS = Homo sapiens] | Up |
| ENO1 | alpha-enolase [OS = Homo sapiens] | Up |
| TYMP; SCO2 | thymidine phosphorylase [OS = Homo sapiens] | Up |
| GLIPR2 | Golgi-associated plant pathogenesis-related protein 1 | Up |
| | [OS = Homo sapiens] | |
| APOE | Apolipoprotein E [OS = Homo sapiens] | Up |
| DCD | Dermcidin [OS = Homo sapiens] | Up |
| KRT78 | Keratin, type II cytoskeletal 78 [OS = Homo sapiens] | Up |
| AKAP9 | Isoform 2 of A-kinase anchor protein 9 [OS = Homo sapiens] | Up |
| GPLD1 | Phosphatidylinositol-glycan-specific phospholipase D [OS = Homo | Up |
| | sapiens] | |
| TXN | thioredoxin [OS = Homo sapiens] | Up |
| IGKC | immunoglobulin kappa constant [OS = Homo sapiens] | Up |
| PRKDC | DNA-dependent protein kinase catalytic subunit [OS = Homo | Up |
| | sapiens] | |
| OIT3 | Oncoprotein-induced transcript 3 protein [OS = Homo sapiens] | Up |
| ZC3H12C | Probable ribonuclease ZC3H12C [OS = Homo sapiens] | Up |
| IGLV1-51 | immunoglobulin lambda variable 1-51 [OS = Homo sapiens] | Up |
| FGR | Tyrosine-protein kinase Fgr [OS = Homo sapiens] | Up |
| AMBP | Protein AMBP [OS = Homo sapiens] | Up |
| C1RL | Complement C1r subcomponent-like protein [OS = Homo | Up |
| | sapiens] | |
| CALM1 | Calmodulin [OS = Homo sapiens] | Up |
| IGLC7 | immunoglobulin lambda constant 7 [OS = Homo sapiens] | Up |
| SAA4 | Serum amyloid A-4 protein [OS = Homo sapiens] | Up |
| HBA1 | Hemoglobin subunit alpha [OS = Homo sapiens] | Up |
| GTF3C1 | General transcription factor 3C polypeptide 1 [OS = Homo | Up |
| | sapiens] | |
| F5 | Coagulation factor V [OS = Homo sapiens] | Up |
| ITIH4 | Inter-alpha-trypsin inhibitor heavy chain H4 [OS = Homo sapiens] | Up |
| DSP | Desmoplakin [OS = Homo sapiens] | Up |
| RASGEF1C | ras-GEF domain-containing family member 1C [OS = Homo | Up |
| | sapiens] | |
| UBTD1 | Ubiquitin domain-containing protein 1 [OS = Homo sapiens] | Up |
| HSPA1A | heat shock 70 kDa protein 1A [OS = Homo sapiens] | Up |
| PRMT8 | Isoform 2 of Protein arginine N-methyltransferase 8 [OS = Homo | Up |
| | sapiens] | |
| CFHR4 | Complement factor H-related protein 4 [OS = Homo sapiens] | Up |
| CENPE | Centromere-associated protein E [OS = Homo sapiens] | Up |
| C2 | complement C2 [OS = Homo sapiens] | Up |
| APOF | Apolipoprotein F [OS = Homo sapiens] | Up |
| BEND3 | BEN domain-containing protein 3 [OS = Homo sapiens] | Up |
| CD14 | Monocyte differentiation antigen CD14 [OS = Homo sapiens] | Up |
| ORM1 | Alpha-1-acid glycoprotein 1 [OS = Homo sapiens] | Up |
| UBA52 | Ubiquitin-60S ribosomal protein L40 [OS = Homo sapiens] | Up |
| KRT9 | Keratin, type I cytoskeletal 9 [OS = Homo sapiens] | Up |
| PODXL | Isoform 2 of Podocalyxin [OS = Homo sapiens] | Up |
| RAB8A | Ras-related protein Rab-8A [OS = Homo sapiens] | Up |
| VWF | Von Willebrand factor [OS = Homo sapiens] | Up |
| C3 | Complement C3 [OS = Homo sapiens] | Up |
| C7 | Complement component C7 [OS = Homo sapiens] | Up |
| KRT13 | Keratin, type I cytoskeletal 13 [OS = Homo sapiens] | Up |
| ARF3 | ADP-ribosylation factor 3 [OS = Homo sapiens] | Up |
| PCBP1 | Poly(RC)-binding protein 1 [OS = Homo sapiens] | Up |
| STK24 | Serine/threonine-protein kinase 24 [OS = Homo sapiens] | Up |
| FABP1 | Fatty acid-binding protein, liver [OS = Homo sapiens] | Up |
| SLC2A1 | Solute carrier family 2, facilitated glucose transporter member 1 | Up |
| | [OS = Homo sapiens] | |
| SLC29A1 | Equilibrative nucleoside transporter 1 [OS = Homo sapiens] | Up |
| PTPN6 | tyrosine-protein phosphatase non-receptor type 6 [OS = Homo | Up |
| | sapiens] | |
| ZNF775 | Zinc finger protein 775 [OS = Homo sapiens] | Up |
| KRT85 | Keratin, type II cuticular Hb5 [OS = Homo sapiens] | Up |
| GNG5 | guanine nucleotide-binding protein g(i)/g(s)/g(o) subunit | Up |
| | gamma-5 [OS = Homo sapiens] | |
| GYPA | Glycophorin-A [OS = Homo sapiens] | Up |
| ATP5A1 | ATP synthase subunit alpha, mitochondrial [OS = Homo sapiens] | Up |
| FLOT2 | Flotillin-2 [OS = Homo sapiens] | Up |
| CD151 | CD151 antigen [OS = Homo sapiens] | Up |
| PCSK9 | Proprotein convertase subtilisin/kexin type 9 [OS = Homo | Up |
| | sapiens] | |
| EDEM3 | ER degradation-enhancing alpha-mannosidase-like protein 3 | Up |
| | [OS = Homo sapiens] | |
| LYN | Isoform 2 of Tyrosine-protein kinase Lyn [OS = Homo sapiens] | Up |
| SERPINB12 | Serpin B12 [OS = Homo sapiens] | Up |
| KIF15 | Kinesin-like protein KIF15 [OS = Homo sapiens] | Up |
| REEP5 | Receptor expression-enhancing protein 5 [OS = Homo sapiens] | Up |
| ZYX | Zyxin [OS = Homo sapiens] | Up |
| IGKV6-21 | Immunoglobulin kappa variable 6-21 [OS = Homo sapiens] | Up |
| FGL1 | Fibrinogen-like protein 1 [OS = Homo sapiens] | Up |
| PCOLCE | Procollagen C-endopeptidase enhancer 1 [OS = Homo sapiens] | Up |
| ATP5B | ATP synthase subunit beta, mitochondrial [OS = Homo sapiens] | Up |
| CD47 | Leukocyte surface antigen CD47 [OS = Homo sapiens] | Up |
| MGP | matrix Gla protein [OS = Homo sapiens] | Up |
| CCT6A | Isoform 2 of T-complex protein 1 subunit zeta [OS = Homo | Up |
| | sapiens] | |
| CDH1 | Cadherin-1 [OS = Homo sapiens] | Up |
| CORO1C | Isoform 3 of Coronin-1C [OS = Homo sapiens] | Up |
| APMAP | Adipocyte plasma membrane-associated protein [OS = Homo | Up |
| | sapiens] | |
| EPS8 | Epidermal growth factor receptor kinase substrate 8 [OS = Homo | Up |
| | sapiens] | |
| RAB5B | Ras-related protein Rab-5B [OS = Homo sapiens] | Up |
| LMAN2 | Vesicular integral-membrane protein VIP36 [OS = Homo sapiens] | Up |
| PSMA3 | Isoform 2 of Proteasome subunit alpha type-3 [OS = Homo | Up |
| | sapiens] | |
| TFIP11 | Tuftelin-interacting protein 11 [OS = Homo sapiens] | Up |
| KRT15 | Keratin, type I cytoskeletal 15 [OS = Homo sapiens] | Up |
| BCAP31 | B-cell receptor-associated protein 31 [OS = Homo sapiens] | Up |
| GPRC5C | G-protein coupled receptor family C group 5 member C | Up |
| | [OS = Homo sapiens] | |
| HSPB1 | Heat shock protein beta-1 [OS = Homo sapiens] | Up |
| APEH | Acylamino-acid-releasing enzyme [OS = Homo sapiens] | Up |
| TPM1 | Isoform 5 of Tropomyosin alpha-1 chain [OS = Homo sapiens] | Up |
| PSMB4 | Proteasome subunit beta type-4 [OS = Homo sapiens] | Up |
| GNB2 | Guanine nucleotide-binding protein G(I)/G(S)/G(T) subunit beta- | Up |
| | 2 [OS = Homo sapiens] | |
| CAP1 | adenylyl cyclase-associated protein 1 [OS = Homo sapiens] | Up |
| ILK | Integrin-linked protein kinase [OS = Homo sapiens] | Up |
| SH3BGRL3 | SH3 domain-binding glutamic acid-rich-like protein 3 [OS = Homo | Up |
| | sapiens] | |
| ASPM | Abnormal spindle-like microcephaly-associated protein | Up |
| | [OS = Homo sapiens] | |
| ASPN | Asporin [OS = Homo sapiens] | Up |
| TPM4 | Tropomyosin alpha-4 chain [OS = Homo sapiens] | Up |
| KDM5A | Lysine-specific demethylase 5A [OS = Homo sapiens] | Up |
| PLEK | pleckstrin [OS = Homo sapiens] | Up |
| ANPEP | aminopeptidase N [OS = Homo sapiens] | Up |
| TMOD1 | Tropomodulin-1 [OS = Homo sapiens] | Up |
| MUC5B | Mucin-5B [OS = Homo sapiens] | Up |
| PECAM1 | Isoform Delta13 of Platelet endothelial cell adhesion molecule | Up |
| | [OS = Homo sapiens] | |
| IGHV3-30 | Immunoglobulin heavy variable 3-30 [OS = Homo sapiens] | Up |
| PPBP | Platelet basic protein [OS = Homo sapiens] | Up |
| F11R | Junctional adhesion molecule A [OS = Homo sapiens] | Up |
| BLVRB | flavin reductase (NADPH) [OS = Homo sapiens] | Up |
| LIMS1 | LIM and senescent cell antigen-like-containing domain protein 1 | Up |
| | [OS = Homo sapiens] | |
| EPB42 | Isoform Long of Erythrocyte membrane protein band 4.2 | Up |
| | [OS = Homo sapiens] | |
| YWHAB | 14-3-3 protein beta/alpha [OS = Homo sapiens] | Up |
| DPP4 | Dipeptidyl peptidase 4 [OS = Homo sapiens] | Up |
| CAT | catalase [OS = Homo sapiens] | Up |
| ANK1 | ankyrin-1 [OS = Homo sapiens] | Up |
| IGHV1-18 | Immunoglobulin heavy variable 1-18 [OS = Homo sapiens] | Up |
| OR5M8 | Olfactory receptor 5M8 [OS = Homo sapiens] | Up |
| SPTA1 | spectrin alpha chain, erythrocytic 1 [OS = Homo sapiens] | Up |
| MPP1 | 55 kDa erythrocyte membrane protein [OS = Homo sapiens] | Up |
| AGT | Angiotensinogen [OS = Homo sapiens] | Up |
| CAPNS1 | Calpain small subunit 1 [OS = Homo sapiens] | Up |
| SLC4A1 | Band 3 anion transport protein [OS = Homo sapiens] | Up |
| PKM | Pyruvate kinase PKM [OS = Homo sapiens] | Up |
| KRT16 | Keratin, type 1 cytoskeletal 16 [OS = Homo sapiens] | Up |
| F13B | Coagulation factor XIII B chain [OS = Homo sapiens] | Up |
| PPIA | peptidyl-prolyl cis-trans isomerase A [OS = Homo sapiens] | Up |
| RTN4 | Reticulon-4 [OS = Homo sapiens] | Up |
| S100A8 | Protein S100-A8 [OS = Homo sapiens] | Up |
| FBLN1 | Fibulin-1 [OS = Homo sapiens] | Up |
| AZGP1 | Zinc-alpha-2-glycoprotein [OS = Homo sapiens] | Down |
| RAP1B | Isoform 3 of Ras-related protein Rap-1b [OS = Homo sapiens] | Down |
| TF | Serotransferrin [OS = Homo sapiens] | Down |
| EEF2 | Elongation factor 2 [OS = Homo sapiens] | Down |
| CCDC168 | Coiled-coil domain-containing protein 168 [OS = Homo sapiens] | Down |
| APOH | Beta-2-glycoprotein 1 [OS = Homo sapiens] | Down |
| APOA5 | Apolipoprotein A-V [OS = Homo sapiens] | Down |
| THBS1 | thrombospondin-1 [OS = Homo sapiens] | Down |
| AHSG | Alpha-2-HS-glycoprotein [OS = Homo sapiens] | Down |
| LRG1 | Leucine-rich alpha-2-glycoprotein [OS = Homo sapiens] | Down |
| APOA1 | Apolipoprotein A-I [OS = Homo sapiens] | Down |
| HTRA1 | Serine protease HTRA1 [OS = Homo sapiens] | Down |
| SPTB | Spectrin beta chain, erythrocytic [OS = Homo sapiens] | Down |
| HTRA2 | Serine protease HTRA2, mitochondrial [OS = Homo sapiens] | Down |
| HBB | Hemoglobin subunit beta [OS = Homo sapiens] | Down |
| EPB41 | Isoform 2 of Protein 4.1 [OS = Homo sapiens] | Down |
| IGKV4-1 | immunoglobulin kappa variable 4-1 [OS = Homo sapiens] | Down |
| RBP4 | Retinol-binding protein 4 [OS = Homo sapiens] | Down |
| SFN | 14-3-3 protein sigma [OS = Homo sapiens] | Down |
| LDHA | L-lactate dehydrogenase A chain [OS = Homo sapiens] | Down |
| APOB | apolipoprotein B-100 [OS = Homo sapiens] | Down |
| TUBA1A | tubulin alpha-1A chain [OS = Homo sapiens] | Down |
| IGHV4-34 | immunoglobulin heavy variable 4-34 [OS = Homo sapiens] | Down |
| C5orf42 | Protein JBTS17 [OS = Homo sapiens] | Down |
| EMILIN1 | EMILIN-1 [OS = Homo sapiens] | Down |
| OR5M8 | Olfactory receptor 5M8 [OS = Homo sapiens] | Down |
| ACTR3 | actin-related protein 3 [OS = Homo sapiens] | Down |
| PSMD2 | 26S proteasome non-ATPase regulatory subunit 2 [OS = Homo | Down |
| | sapiens] | |
| MYL6 | Isoform Smooth muscle of Myosin light polypeptide 6 | Down |
| | [OS = Homo sapiens] | |
| TKT | Transketolase [OS = Homo sapiens] | Down |
| APOC4 | Apolipoprotein C-IV [OS = Homo sapiens] | Down |
| CCT2 | T-complex protein 1 subunit beta [OS = Homo sapiens] | Down |
| MYO15A | Unconventional myosin-XV [OS = Homo sapiens] | Down |
| SERPINA4 | Kallistatin [OS = Homo sapiens] | Down |
| ARPC5 | Actin-related protein 2/3 complex subunit 5 [OS = Homo sapiens] | Down |
| NCKAP5L | Nck-associated protein 5-like [OS = Homo sapiens] | Down |
| HRG | Histidine-rich glycoprotein [OS = Homo sapiens] | Down |
| RAP2B | ras-related protein Rap-2b [OS = Homo sapiens] | Down |
| CORO1A | Coronin-1A [OS = Homo sapiens] | Down |
| CD44 | Isoform 12 of CD44 antigen [OS = Homo sapiens] | Down |
| HSPG2 | Basement membrane-specific heparan sulfate proteoglycan | Down |
| | core protein | |
| | [OS = Homo sapiens] | |
| APOA4 | Apolipoprotein A-IV [OS = Homo sapiens] | Down |
| KPRP | Keratinocyte proline-rich protein [OS = Homo sapiens] | Down |
| RAB11A | Isoform 2 of Ras-related protein Rab-11A [OS = Homo sapiens] | Down |
| PPBP | Platelet basic protein [OS = Homo sapiens] | Down |
| SCUBE2 | Signal peptide, CUB and EGF-like domain-containing protein 2 | Down |
| | [OS = Homo sapiens] | |
| C1orf56 | Protein MENT [OS = Homo sapiens] | Down |
| C5orf46 | Uncharacterized protein C5orf46 [OS = Homo sapiens] | Down |
| S100A16 | Protein S100-A16 [OS = Homo sapiens] | Down |
| MUC1 | mucin-1 [OS = Homo sapiens] | Down |
| TSKU | Tsukushin [OS = Homo sapiens] | Down |
| IGKV2-24 | immunoglobulin kappa variable 2-24 [OS = Homo sapiens] | Down |
| HABP2 | Hyaluronan-binding protein 2 [OS = Homo sapiens] | Down |
| RHOG | Rho-related GTP-binding protein RhoG [OS = Homo sapiens] | Down |
| CALD1 | Isoform 6 of Caldesmon [OS = Homo sapiens] | Down |
| BCHE | cholinesterase [OS = Homo sapiens] | Down |
| CNP | 2′,3′-cyclic-nucleotide 3′-phosphodiesterase [OS = Homo sapiens] | Down |
| WDR1 | WD repeat-containing protein 1 [OS = Homo sapiens] | Down |
| SLC2A3 | Solute carrier family 2, facilitated glucose transporter member 3 | Down |
| | [OS = Homo sapiens] | |
| SLC14A1 | Isoform 2 of Urea transporter 1 [OS = Homo sapiens] | Down |
| PIEZO1 | Piezo-type mechanosensitive ion channel component 1 | Down |
| | [OS = Homo sapiens] | |
| HLA-DRB5 | HLA class II histocompatibility antigen, DR beta 5 chain | Down |
| | [OS = Homo sapiens] | |
| EP400 | E1A-binding protein p400 [OS = Homo sapiens] | Down |
| KCNG1 | Potassium voltage-gated channel subfamily G member 1 | Down |
| | [OS = Homo sapiens] | |
| IGLV2-8 | Immunoglobulin lambda variable 2-8 [OS = Homo sapiens] | Down |
| CD59 | CD59 glycoprotein [OS = Homo sapiens] | Down |
| CHGA | Chromogranin-A [OS = Homo sapiens] | Down |
| SCLT1 | Sodium channel and clathrin linker 1 [OS = Homo sapiens] | Down |
| IGHV1OR15-1 | Immunoglobulin heavy variable 1-2 [OS = Homo sapiens] | Down |
| EDEM3 | ER degradation-enhancing alpha-mannosidase-like protein 3 | Down |
| | [OS = Homo sapiens] | |
| ADH4 | Isoform 2 of Alcohol dehydrogenase 4 [OS = Homo sapiens] | Down |
| C4A; C4B | Complement C4-A [OS = Homo sapiens] | Down |
| IGKV2D-29 | immunoglobulin kappa variable 2D-29 [OS = Homo sapiens] | Down |
| IGHV3-64D | immunoglobulin heavy variable 3-64D [OS = Homo sapiens] | Down |
| SOD1 | Superoxide dismutase [Cu—Zn] [OS = Homo sapiens] | Down |
| KRT15 | Keratin, type I cytoskeletal 15 [OS = Homo sapiens] | Down |
| CUL4B | Cullin-4B [OS = Homo sapiens] | Down |
| ZGRF1 | Protein ZGRF1 [OS = Homo sapiens] | Down |
| IGHA2 | Immunoglobulin alpha-2 heavy chain [OS = Homo sapiens] | Down |
| PZP | Pregnancy zone protein [OS = Homo sapiens] | Down |
| SBSN | Isoform 2 of Suprabasin [OS = Homo sapiens] | Down |
| ADIRF | Adipogenesis regulatory factor [OS = Homo sapiens] | Down |
| EFEMP1 | EGF-containing fibulin-like extracellular matrix protein 1 | Down |
| | [OS = Homo sapiens] | |
| PSAP | Prosaposin [OS = Homo sapiens] | Down |
| VIM | Vimentin [OS = Homo sapiens] | Down |
| CROCC | Rootletin [OS = Homo sapiens] | Down |
| DESI1 | Desumoylating isopeptidase 1 [OS = Homo sapiens] | Down |
| SLC25A18 | Mitochondrial glutamate carrier 2 [OS = Homo sapiens] | Down |
| HPR | Haptoglobin-related protein [OS = Homo sapiens] | Down |
| COL6A3 | Isoform 4 of Collagen alpha-3(VI) chain [OS = Homo sapiens] | Down |
| CAPG | Macrophage-capping protein [OS = Homo sapiens] | Down |
| AHNAK2 | Protein AHNAK2 [OS = Homo sapiens] | Down |
| BAIAP2 | Isoform 3 of Brain-specific angiogenesis inhibitor 1-associated | Down |
| | protein 2 | |
| | [OS = Homo sapiens] | |
| A1BG | Alpha-lB-glycoprotein [OS = Homo sapiens] | Down |
| IGHV4-4 | immunoglobulin heavy variable 4-4 [OS = Homo sapiens] | Down |
| ATRN | Attractin [OS = Homo sapiens] | Down |
| DNM2 | Dynamin-2 [OS = Homo sapiens] | Down |
| ZNF521 | Zinc finger protein 521 [OS = Homo sapiens] | Down |
| PF4 | Platelet factor 4 [OS = Homo sapiens] | Down |
| RAB1B | ras-related protein Rab-1B [OS = Homo sapiens] | Down |
| ZSWIM9 | Uncharacterized protein ZSWIM9 [OS = Homo sapiens] | Down |
| PPP2R1A | serine/threonine-protein phosphatase 2A 65 kDa regulatory | Down |
| | subunit A alpha isoform | |
| RBM11 | Splicing regulator RBM11 [OS = Homo sapiens] | Down |
| CEP120 | Centrosomal protein of 120 kDa [OS = Homo sapiens] | Down |
| PFN1 | profilin-1 [OS = Homo sapiens] | Down |
| IGLV2-11 | Immunoglobulin lambda variable 2-11 [OS = Homo sapiens] | Down |
| PIBF1 | Progesterone-induced-blocking factor 1 [OS = Homo sapiens] | Down |
| PEX5 | Peroxisomal targeting signal 1 receptor [OS = Homo sapiens] | Down |
| GP1BA | Platelet glycoprotein Ib alpha chain [OS = Homo sapiens] | Down |
| PTPRC | Isoform 2 of Receptor-type tyrosine-protein phosphatase C | Down |
| | [OS = Homo sapiens] | |
| KRT3 | Keratin, type II cytoskeletal 3 [OS = Homo sapiens] | Down |
| CRTAC1 | cartilage acidic protein 1 [OS = Homo sapiens] | Down |
| PRSS56 | Serine protease 56 [OS = Homo sapiens] | Down |
| ALDH9A1 | 4-trimethylaminobutyraldehyde dehydrogenase [OS = Homo | Down |
| | sapiens] | |
| VNN1 | Pantetheinase [OS = Homo sapiens] | Down |
| MTO1 | Protein MTO1 homolog, mitochondrial [OS = Homo sapiens] | Down |
| MLNR | Motilin receptor [OS = Homo sapiens] | Down |
| LYZ | lysozyme c [OS = Homo sapiens] | Down |
| ABCC5 | Multidrug resistance-associated protein 5 [OS = Homo sapiens] | Down |
| PCLO | Isoform 6 of Protein piccolo [OS = Homo sapiens] | Down |
| ZNF518A | Zinc finger protein 518A [OS = Homo sapiens] | Down |
| IGKV3D-20 | immunoglobulin kappa variable 3D-20 [OS = Homo sapiens] | Down |
| CD36 | Platelet glycoprotein 4 [OS = Homo sapiens] | Down |
| NPM1 | Nucleophosmin [OS = Homo sapiens] | Down |
| CD55 | Isoform 5 of Complement decay-accelerating factor [OS = Homo | Down |
| | sapiens] | |
| TALDO1 | Transaldolase [OS = Homo sapiens] | Down |
| CD47 | Leukocyte surface antigen CD47 [OS = Homo sapiens] | Down |
| C20orf96 | Uncharacterized protein C20orf96 [OS = Homo sapiens] | Down |
| IGLV3-10 | immunoglobulin lambda variable 3-10 [OS = Homo sapiens] | Down |
| MCTS1 | Isoform 2 of Malignant T-cell-amplified sequence 1 [OS = Homo | Down |
| | sapiens] | |
| MSL1 | male-specific lethal 1 homolog [OS = Homo sapiens] | Down |
| PLSCR4 | Phospholipid scramblase 4 [OS = Homo sapiens] | Down |
| ADAMTSL2 | ADAMTS-like protein 2 [OS = Homo sapiens] | Down |
| CPE | Carboxypeptidase E [OS = Homo sapiens] | Down |
| ADD2 | Beta-adducin [OS = Homo sapiens] | Down |
| HSPA4 | Heat shock 70 kDa protein 4 [OS = Homo sapiens] | Down |
| HAUS6 | HAUS augmin-like complex subunit 6 [OS = Homo sapiens] | Down |
| ARPC1B | Actin-related protein 2/3 complex subunit 1B [OS = Homo | Down |
| | sapiens] | |
| IGKV3D-11 | immunoglobulin kappa variable 3D-11 [OS = Homo sapiens] | Down |
| IGLV3-27 | immunoglobulin lambda variable 3-27 [OS = Homo sapiens] | Down |
| PPIB | peptidyl-prolyl cis-trans isomerase B [OS = Homo sapiens] | Down |
| EMC4 | ER membrane protein complex subunit 4 [OS = Homo sapiens] | Down |
| CCL18 | C-C motif chemokine 18 [OS = Homo sapiens] | Down |
| IGKV3-20 | Immunoglobulin kappa variable 3-20 [OS = Homo sapiens] | Down |
| PSMA5 | Proteasome subunit alpha type-5 [OS = Homo sapiens] | Down |
| DBT | Lipoamide acyltransferase component of branched-chain alpha- | Down |
| | keto acid dehydrogenase | |
| ME1 | NADP-dependent malic enzyme | Down |
| FAM114A2 | Protein FAM114A2 [OS = Homo sapiens] | Down |
| ICK | Serine/threonine-protein kinase ICK [OS = Homo sapiens] | Down |
| YWHAH | 14-3-3 protein eta [OS = Homo sapiens] | Down |
| MYL12B | Myosin regulatory light chain 12B [OS = Homo sapiens] | Down |
| CHMP3 | Charged multivesicular body protein 3 [OS = Homo sapiens] | Down |
| KRT76 | Keratin, type II cytoskeletal 2 oral [OS = Homo sapiens] | Down |
| PNMA6A | Paraneoplastic antigen-like protein 6A [OS = Homo sapiens] | Down |
| KRT23 | Keratin, type I cytoskeletal 23 [OS = Homo sapiens] | Down |
| PBXIP1 | Pre-B-cell leukemia transcription factor-interacting protein 1 | Down |
| | [OS = Homo sapiens] | |
| BNC2 | Zinc finger protein basonuclin-2 [OS = Homo sapiens] | Down |
| SH3RF2 | Putative E3 ubiquitin-protein ligase SH3RF2 [OS = Homo sapiens] | Down |
| IGLV4-69 | immunoglobulin lambda variable 4-69 [OS = Homo sapiens] | Down |
| IGLV7-46 | Immunoglobulin lambda variable 7-46 [OS = Homo sapiens] | Down |
| PTGS1 | Prostaglandin G/H synthase 1 [OS = Homo sapiens] | Down |
| VAPB | Vesicle-associated membrane protein-associated protein B/C | Down |
| | [OS = Homo sapiens] | |
| IGLV2-14 | Immunoglobulin lambda variable 2-14 [OS = Homo sapiens] | Down |
| CC2D2B | Protein CC2D2B [OS = Homo sapiens] | Down |
| CHMP4B | Charged multivesicular body protein 4b [OS = Homo sapiens] | Down |
| SERPIND1 | Heparin cofactor 2 [OS = Homo sapiens] | Down |
| PGK1 | phosphoglycerate kinase 1 [OS = Homo sapiens] | Down |
| PON1 | Serum paraoxonase/arylesterase 1 [OS = Homo sapiens] | Down |
| GC | Isoform 3 of Vitamin D-binding protein [OS = Homo sapiens] | Down |
| PLG | Plasminogen [OS = Homo sapiens] | Down |
| TTR | Transthyretin [OS = Homo sapiens] | Down |
| ANXA2 | Annexin A2 [OS = Homo sapiens] | Down |
| ANXA1 | annexin A1 [OS = Homo sapiens] | Down |
| IGHG4 | Immunoglobulin heavy constant gamma 4 [OS = Homo sapiens] | Down |
| SERPINF1 | Pigment epithelium-derived factor [OS = Homo sapiens] | Down |
| MFGE8 | Lactadherin [OS = Homo sapiens] | Down |
| LTBP1 | Isoform 4 of Latent-transforming growth factor beta-binding | Down |
| | protein 1 | |
| | [OS = Homo sapiens] | |
| ITGA6 | Isoform Alpha-6X1X2A of Integrin alpha-6 [OS = Homo sapiens] | Down |
| LTF | Isoform DeltaLf of Lactotransferrin [OS = Homo sapiens] | Down |
| ADAMTSL4 | ADAMTS-like protein 4 [OS = Homo sapiens] | Down |
| IDE | insulin-degrading enzyme [OS = Homo sapiens] | Down |
| PTPN23 | Tyrosine-protein phosphatase non-receptor type 23 [OS = Homo | Down |
| | sapiens] | |
| TUBB | tubulin beta chain [OS = Homo sapiens] | Down |
| IGHV3-43 | Immunoglobulin heavy variable 3-43 [OS = Homo sapiens] | Down |
| DSC1 | Isoform 1B of Desmocollin-1 [OS = Homo sapiens] | Down |
| HSP90B1 | Endoplasmin [OS = Homo sapiens] | Down |
| CFP | Properdin [OS = Homo sapiens] | Down |
| ZNF292 | zinc finger protein 292 [OS = Homo sapiens] | Down |
| CHST9 | carbohydrate sulfotransferase 9 [OS = Homo sapiens] | Down |
| MASP1 | Isoform 2 of Mannan-binding lectin serine protease 1 | Down |
| | [OS = Homo sapiens] | |
| ALDH1A1 | Retinal dehydrogenase 1 [OS = Homo sapiens] | Down |
| CPN1 | Carboxypeptidase N catalytic chain [OS = Homo sapiens] | Down |
| HIST1H4A | histone H4 [OS = Homo sapiens] | Down |
| PSG1 | Isoform 4 of Pregnancy-specific beta-1-glycoprotein 1 | Down |
| | [OS = Homo sapiens] | |
| SYCE1 | synaptonemal complex central element protein 1 [OS = Homo | Down |
| | sapiens] | |
| SERPINB4 | Serpin B4 [OS = Homo sapiens] | Down |
| UXS1 | UDP-glucuronic acid decarboxylase 1 [OS = Homo sapiens] | Down |
| SHPRH | E3 ubiquitin-protein ligase SHPRH [OS = Homo sapiens] | Down |
| PNPLA8 | Calcium-independent phospholipase A2-gamma [OS = Homo | Down |
| | sapiens] | |
| IGLV9-49 | immunoglobulin lambda variable 9-49 [OS = Homo sapiens] | Down |
| SIRPB1 | Signal-regulatory protein beta-1 isoform 3 [OS = Homo sapiens] | Down |
| CLIC4 | Chloride intracellular channel protein 4 [OS = Homo sapiens] | Down |
| SCIN | ADSEVERIN [OS = Homo sapiens] | Down |
| LGALS1 | Galectin-1 [OS = Homo sapiens] | Down |
| LGALS7 | galectin-7 [OS = Homo sapiens] | Down |
| S100A4 | Protein S100-A4 [OS = Homo sapiens] | Down |
| POSTN | Periostin [OS = Homo sapiens] | Down |
| CPA4 | Carboxypeptidase A4 [OS = Homo sapiens] | Down |
| MAP2K7 | dual specificity mitogen-activated protein kinase kinase 7 | Down |
| | [OS = Homo sapiens] | |
| TRDMT1 | tRNA (cytosine(38)-C(5))-methyltransferase [OS = Homo sapiens] | Down |
| CRIP2 | Cysteine-rich protein 2 [OS = Homo sapiens] | Down |
| TPP2 | Tripeptidyl-peptidase 2 [OS = Homo sapiens] | Down |
| CAND1 | cullin-associated nedd8-dissociated protein 1 [OS = Homo | Down |
| | sapiens] | |
| COG5 | Isoform 3 of Conserved oligomeric Golgi complex subunit 5 | Down |
| | [OS = Homo sapiens] | |
| ANXA7 | Annexin A7 [OS = Homo sapiens] | Down |
| IGHV2-5 | immunoglobulin heavy variable 2-5 [OS = Homo sapiens] | Down |
| C10orf90 | Centrosomal protein C10orf90 [OS = Homo sapiens] | Down |
| S100A14 | protein S100-A14 [OS = Homo sapiens] | Down |
| DSC3 | Isoform 3B of Desmocollin-3 [OS = Homo sapiens] | Down |

While this disclosure has been described as having an exemplary design, the present disclosure may be further modified within the spirit and scope of this disclosure. This application is therefore intended to cover any variations, uses, or adaptations of the disclosure using its general principles. Further, this application is intended to cover such departures from the present disclosure as come within known or customary practice in the art to which this disclosure pertains.

What is claimed is:

1. A compound comprising:
a biomarker for pancreatic cancers, the biomarker selected from the group consisting of extracellular vesicle (EV) proteins and any combination thereof, wherein each of the EV proteins or their combinations are capable of differentiating a human with pancreatic ductal adenocarcinoma (PDAC) from a healthy human and a human with non-cancer conditions, for the purposes of PDAC diagnosis, prognosis, detection, monitoring, patient stratification, drug response analysis, therapy selection, or the like.

2. The compound of claim 1, wherein the biomarker has a putative compound identification, match form, name or pathway.

3. The compound of claim 1, wherein the biomarker is located on, in or about an extracellular vesicle.

4. The compound of claim 3, wherein the extracellular vesicle including the biomarker is captured, enriched or isolated using a method for capture, enrichment or isolation of extracellular vesicles.

5. The compound of claim 4, wherein the method for capture, enrichment or isolation of extracellular vesicles is selected from the group consisting of Extracellular Vesicles total recovery and purification (EVtrap), ultracentrifugation (UC), filtrations, antibody-based purification, size-exclusion approach, polymer precipitation and affinity capture.

6. The compound of claim 3, wherein the biomarker is detected from a human's biofluid.

7. The compound of claim 6, wherein the biofluid is selected from the group consisting of plasma and serum.

8. The compound of claim 6, wherein the biomarker is selected from a pre-determined biomarkers panel.

9. The compound of claim 3, wherein the extracellular vesicle is an exosome, microvesicle, endosome or other extracellular vesicle.

10. A method of detecting biomarkers comprising the steps of:
analyzing blood samples from humans with pancreatic ductal adenocarcinoma (PDAC), humans with chronic pancreatitis of different etiologies, humans with intraductal papillary mucinous neoplasm (IPMN), or other relevant conditions for an extracellular vesicle (EV) biomarker; and
detecting a biomarker in blood sample for the purposes of PDAC diagnosis, prognosis, detection, monitoring, patient stratification, drug response analysis, therapy selection, or the like, wherein the biomarker consists of blood EV proteins and any combination thereof.

11. The method of claim 10, further comprising the step of:
analyzing differences in detected biomarkers between cancer and non-cancer blood samples, including observing that an EV proteomics of humans having PDAC cancer has clear separation from an EV proteomics of humans having non-cancer conditions or healthy controls.

12. The method of claim 11, further comprising the step of:
assessing the predictive capacity of detected biomarkers.

13. The method of claim 10, further comprising the step of:
identification of novel biomarkers.

14. The method of claim 10, wherein the biomarkers are selected from a pre-determined biomarkers panel.

15. The method of claim 10, wherein the step of analyzing blood samples further includes processing and enrichment of EVs, filtering out soluble proteins and retaining EV associated proteins, wherein the protein profiles in EV concentrates are different from protein profiles naturally occurring in the blood samples.

16. The method of claim 15, wherein the step of analyzing blood samples further includes enrichment of immune-, complement- and coagulation-related proteins from the EV proteome in humans having PDAC, chronic pancreatitis of different etiologies, intraductal papillary mucinous neoplasm (IPMN) or other relevant conditions.

17. The method of claim 16, further comprising the step of:
performing biostatistical analysis in detected biomarkers between cancer and non-cancer controls including observing that the EV proteomics of humans having PDAC has clear separation from the EV proteomics of humans having non-cancer conditions or healthy controls.

18. The method of claim 17, further comprising the step of:
assessing a disease predictive capacity of detected biomarkers.

19. The method of claim 18, further comprising the step of:
Identification of novel biomarkers.
